(19) United States
(12) Patent Application Publication
(10) Pub. No.: US 2010/0132900 A1
(43) Pub. Date: Jun. 3, 2010

(54) METHOD FOR DETERMINING THE BRIGHTNESS OF PAPER PULP

(76) Inventors: Niclas Andersson, Karlstad (SE); Marcus True, Ravensburg (DE)

Correspondence Address: DICKSTEIN SHAPIRO LLP, 1825 EYE STREET NW, Washington, DC, 20006-5403, US

(21) Appl. No.: 12/513,198

(22) PCT Filed: Nov. 1, 2007
(86) PCT No.: PCT/SE07/00975

§ 371 Date: Jan. 22, 2010

(30) Foreign Application Priority Data
Nov. 1, 2006 (SE) 0602305-5

(51) Int. Cl. G01N 21/64; G01N 33/34; D21H 23/06

(52) U.S. Cl. 162/49

(57) ABSTRACT

The invention refers to a method for determination brightness of paper pulp by measuring fluorescence from UV. The measurement is carried out directly in the pipe in which the paper pulp, which shall constitute the paper, is fed forward, by an in-line brightness meter, which also is capable to measure fluorescent substances in the paper pulp, a direct control of the partial stages in the paper pulp process where fluorescent substances can exist or a determination at the end of the process can be accomplished.

The present invention refers to a method for determination of brightness of paper pulp by measuring of fluorescence from UV.

When manufacturing paper pulp for further manufacturing of paper, a sequence of partial steps is used, where the characteristics of the product are developed and determined. During bleaching paper pulp typically a number of process steps are used—several chemical charges/bleaching towers, extraction stages and washing stages—in order to achieve the desired brightness. The importance that each stage performs well is great, since it is normally not possible to compensate for too large deviations from the target value in the previous stage. Furthermore, the influence of the process conditions on the characteristics of the paper pulp is such that there are great economical and quality related advantages to work at an optimal operation point.

The dynamics of a paper pulp production process requires in-line instrumentation for continuous measurement of key parameters. The signals from the instruments give the possibility to steer and control the process in order to be able to work with a small variation around the desired working point. Inter alia there are in-line brightness meters, which measure the reflectance of the paper pulp in a process pipe. These meters are considered as indirect since they are calibrated against measurements obtained according to a standard method. When measurements are made off-line, on samples taken for analysis in a laboratory for instance, the process cannot be controlled properly, since the measurement results take too long time to be usably.

In order to increase the apparent brightness supplements are today often used in many products, which are named optical brightening agents, or fluorescent whitening agents. These function so that if the paper is illuminated with UV-light a portion of it is reflected at another, higher wavelength, in the visible range. The phenomenon is called by aid of chemical terms fluorescence. In conventional laboratory analysis of paper sheet today the brightness is measured according a standard either employing illumination containing UV-light or not, depending on if it is desired to get information concerning the contribution from, or content of, fluorescent whiteners or not. There is today no in-line meter for determination of brightness including fluorescent whiteners. Therefore nobody has been able to carry out measurements in-line for determination of brightness including the contribution from fluorescent substances.

The object of the present invention is to use an in-line brightness meter, constructed also to be able to measure fluorescent substances in the paper pulp, for controlling of those partial stages in the paper pulp process, where fluorescent substances are present, or for determination at the end of the process. In this case it concerns measuring of both conventional brightness and brightness including the contribution from fluorescent substances, and calculation of the difference between them as a measure of the content of fluorescent substances. Control parameters can be, depending on the type of process stage: Charging of fluorescent whiteners, charging of bleaching chemicals (together with typical parameters such as temperature, time, pressure, pH etc.), and control of raw material composition at recycle pulp use.

The in-line meters existing today normally use light sources (typically light emitting diodes), which emit light in limited wavelength ranges. The reflected light is then registered using a detector with sensitivity in a larger wavelength range, often from UV to short IR wavelengths. The relation between the measured reflectance when e.g. only one blue light source is used (R_{B}) and the brightness (L) then can be

L=k_{B}·R_{B} [Eq. 1]

where k_{B} is a calibration constant.

When measuring brightness including (UV-) fluorescent substances (L*) the corresponding relation

L*=k_{B}·R_{B}+k_{UV}·R_{UV} [Eq. 2]

can be used, where k_{UV} is a calibrating constant for the relation between the reflectance (fluorescence) of the UV-light and that part of the brightness (L*), which originatrs from fluorescent substances. A prerequisite for a good functionality is that a measurement also is made using UV-light (preferably sequentially with other wave-lengths/light sources), corresponding to the reflectance R_{UV} in Eq. 2, and that it is ensured that the detector does not measure the light, which is reflected in the UV-range, but only that, which by fluorescence is reflected at a higher (visible) wavelength. This can be accomplished with the detector stated above by placing an optical filter, which is blocking UV-light, in front of the detector.

Alternatively a spectrometer can be used i.e. a wavelength sensitive detector, where the signal in the visible part of the spectrum is used as a measure of the brightness in a corresponding way.

1. A method for determination brightness of paper pulp by measuring fluorescence from UV, characterized in that the measurement is carried out directly in the pipe the paper pulp, which shall constitute the paper, is fed forward in by an in-line brightness meter which also is capable to measure fluorescent substances in the paper pulp, a direct control of these partial stages in the paper pulp process where fluorescent substances can exist or a determination at the end of the process be accomplished.

2. A method according to claim 1, characterized in that the brightness meter comprises a detector by which only the light is measured which by fluorescence is reflected at a visible wavelength.

3. A method according to claim 2, characterized in that an optical filter blocking UV-light is placed in front of the detector in order to prevent that light which is reflected in the UV-range is measured, so that only light which is reflected by fluorescence at a visible wavelength is measured.

4. A method according to claim 2, characterized in that a spectrometer is used during the measurement, whereby only that light, which by fluorescence is reflected at a visible wavelength, is used as a measure of the brightness.

5. A method according to claim 1, characterized in that the control of the process is made using the brightness including the contribution from fluorescent substances.

6. A method according to claim 1, characterized in that the control of the process is made using the brightness contribution from fluorescent substances, i.e. the difference between the brightness including respectively excluding contribution from fluorescent substances.
